(19) United States
(12) Patent Application Publication
(10) Pub. No.: US 2025/0359576 A1
(43) Pub. Date: Nov. 27, 2025

(54) THEANINE-CONTAINING COMPOSITION

(71) Applicant: TAIYO KAGAKU Co., Ltd., Yokkaichi (JP)

(72) Inventors: Chihiro YAMAMOTO, Yokkaichi (JP); Makoto OZEKI, Yokkaichi (JP)

(21) Appl. No.: 19/212,786

(22) Filed: May 20, 2025

(30) Foreign Application Priority Data
May 21, 2024 (JP) 2024-082272

(51) Int. Cl. A23L 27/00; A23L 27/20; A23L 27/21; A61K 31/198

(52) U.S. Cl. CPC A23L 27/88; A23L 27/202; A23L 27/21; A61K 31/198

(57) ABSTRACT

A theanine-containing composition contains theanine and an organic nitrogen compound. The organic nitrogen compound is preferably at least one selected from the group consisting of amide compounds, imide compounds, and primary amines. More preferably, the organic nitrogen compound is at least one selected from the group consisting of ethylformamide, ethylacetamide, and acetamide.

### CROSS-REFERENCE

The present application claims priority to Japanese patent application no. 2024-082272 filed on May 21, 2024, the contents of which are incorporated herein by reference.

### TECHNICAL FIELD

The invention relates to theanine-containing compositions.

### BACKGROUND ART

Theanine is a component, e.g., of tea extracts, and is known to have physiological effects such as imparting a relaxing effect and improving the quality of sleep. Theanine is also known to be an umami component of tea, and can suppress the bitterness of catechin.

A technique having the goal of altering the taste of theanine is disclosed in JP 2017-012077 A. When high concentrations of theanine are ingested with a beverage, sometimes there is a persistence of a distinctive flavor and an unpleasant aftertaste. JP 2017-012077 A discloses techniques for eliminating this phenomenon and in particular, describes that the taste of theanine can be improved by adding a moderate amount of starch and monosaccharides.

### SUMMARY

Theanine has the potential to enhance the complexity of taste in addition to improving the taste of a food or beverage, so there is potential for further improvements.

It is therefore one non-limiting object of the present teachings to disclose techniques for enhancing the complexity of the taste of a theanine-containing composition.

In one non-limiting aspect of the present teachings, a theanine-containing composition preferably contains (A) theanine and (B) an organic nitrogen compound.

It is preferable that (B) the organic nitrogen compound is at least one compound selected from the group consisting of amide compounds, imide compounds, and primary amines.

More preferably, (B) the organic nitrogen compound is at least one compound selected from the group consisting of ethylformamide, ethylacetamide, and acetamide.

Also, it is preferable that the ratio of ethylformamide to theanine is 5 ppm to 5000 ppm.

In addition or in the alternative, it is preferable that the ratio of ethylacetamide to theanine is 5 ppm to 5000 ppm.

In addition or in the alternative, it is preferable that the ratio of acetamide to theanine is 5 ppm to 5000 ppm.

Foods and drinks according to the present teachings contain such a theanine-containing composition. Representative, non-limiting examples of foods and drinks according to the present teachings include, but are not limited to, beverages (milk-containing drinks, coffee, tea, juice, processed milk, sports drinks, etc.), baked goods (bread, pizza, pies, etc.), sweets (cookies, crackers, biscuits, cakes, castella, etc.), noodles, pasta, snacks, confectionery (candy, caramel, gum, chocolate, etc.), frozen desserts (ice cream, sorbet, etc.), dairy products (cream, cheese, mousse, milk powder, condensed milk, dairy drinks, etc.), pastries (jelly, pudding, mousse, yogurt, butter cream, custard cream, etc.), Japanese sweets (Gyuhi, Uiro, rice cakes, Ohagi, Dorayaki, etc.), processed fruit foods and processed vegetable foods (jams and marmalades, e.g., preserved in syrup, candied fruit, etc.), pastes (flower paste, fruit paste, peanut paste, peanut butter, etc.), seasonings and condiments (soy sauce, Worcestershire sauce, tare sauce, noodle soup base, dashi stock, soup stock, etc.), frozen foods and refrigerated foods (ham, sausage, bacon, hamburger steak, meatballs, croquettes, gyoza, pilaf, rice balls, etc.), processed seafoods (Chikuwa (a type of fish cake), Kamaboko (another type of fish cake), etc.), and health care products such as supplements, and other foods and drinks. Note that foods and drinks according to the present teachings are not limited to foods and drinks for humans only, but also include foods and drinks for pets (dogs, cats, etc.).

In one aspect of the present teachings, the at least one organic nitrogen compound selected from the group consisting of amide compounds, imide compounds, and primary amines includes at least one compound selected from the group consisting of acetamide, ethylacetamide, ethylpropionamide, ethylformamide, succinimide, hydroxysuccinimide, diethyloxamide, and ethylamine, and is preferably ethylformamide, ethylacetamide, and/or acetamide.

According to another aspect of the present teachings, a composition that enhances the complexity of the taste of theanine is provided. In particular, the coexistence of theanine and ethylacetamide have a significant effect on umami flavor and sweetness. The coexistence of theanine and ethylformamide have a significant effect on bitterness. The coexistence of theanine and acetamide have a significant effect on umami flavor.

The terms “umami” and “umami flavor” are intended to mean or refer to a pleasant or pleasing savory taste of a food or beverage. Foods and condiments that are known to have an “umami” flavor include, e.g., meats, shellfish, dashi, tomatoes, mushrooms, soy sauce, fish sauce.

### BRIEF DESCRIPTION OF THE DRAWINGS

FIG. 1 shows the results of a sensory evaluation of solutions containing theanine and organic nitrogen compound A (ethylacetamide).

FIG. 2 shows the results of a sensory evaluation of solutions containing theanine and organic nitrogen compound B (ethylformamide).

FIG. 3 shows the results of a sensory evaluation of solutions containing theanine and organic nitrogen compound C (acetamide).

FIG. 4 shows the results of a sensory evaluation of solutions containing theanine and organic nitrogen compound D (ethylpropionamide).

FIGS. 5A and 5B show the analytical results of the sensory evaluation of solutions containing theanine and organic nitrogen compound A (ethylacetamide) shown in FIG. 1.

FIGS. 6A and 6B show the analytical results of the sensory evaluation of solutions containing theanine and organic nitrogen compound B (ethylformamide) shown in FIG. 2.

FIGS. 7A and 7B show the analytical results of the sensory evaluation of solutions containing theanine and organic nitrogen compound C (acetamide) shown in FIG. 3.

FIG. 8 shows the results of a sensory evaluation of solutions containing organic nitrogen compounds A and C in combination.

FIGS. 9A and 9B show the results of a Student's paired t-test on the samples shown in FIG. 8 in which a significant difference in at least one evaluation category was discerned.

FIG. 10 shows the evaluation results for solutions containing organic nitrogen compound A and C, respectively.

FIG. 11 shows the results of a three-point discrimination sensory evaluation.

### DETAILED DESCRIPTION OF PREFERRED EMBODIMENTS

Next, embodiments of the present teachings will be described with reference to the Figures. The technical scope of the invention is not limited to these embodiments, and various modifications can be made without changing the gist of the invention.

### Production of Theanine

### Example 1: Production of Theanine by an Enzymatic Method

21.9 g of glutamine and 28.5 g of ethylamine hydrochloride were reacted with 0.3 U glutaminase (Amano Pharmaceutical Co., Ltd.) in 0.5 L of 0.05 M borate buffer (pH 9.5) at 30 degrees Celsius for 22 hours. The reaction solution was then subjected to column chromatography using Dowex™ 50×8 ion-exchange resin and Dowex™ 1×2 ion exchange resin (both from Muromachi Chemical Industry Co., Ltd.), and L-theanine was isolated from the reaction solution by using ethanol as the eluent.

The substance was confirmed to be L-theanine by subjecting the isolated substance to an amino acid analyzer and paper chromatography; that is, the isolated substance exhibited the same behavior as standard L-theanine in these analyses. When the isolated substance was hydrolyzed with hydrochloric acid or glutaminase, glutamic acid and ethylamine were produced in a 1:1 molar ratio. The fact that the isolated substance was hydrolyzed by glutaminase demonstrated that ethylamine was bonded to the y-position of glutamic acid. Furthermore, glutamate dehydrogenase was used to confirm that the glutamic acid produced by hydrolysis was the L-form. 8.5 g of L-theanine was obtained thereby.

In the embodiments, theanine produced in Example 1 was used, but theanine obtained by other methods (for example, chemical synthesis, extraction from tea leaves, microbial fermentation, etc.) can be used in accordance with the present invention.

Tests Regarding the Effects of the Combination of Theanine and Various Organic Nitrogen Compounds on Taste

### Test Example 1: Evaluation of the Effects of Each Organic Nitrogen Compound on the Taste of Theanine

Ethylacetamide (organic nitrogen compound A), ethylformamide (organic nitrogen compound B), acetamide (organic nitrogen compound C), and ethylpropionamide (organic nitrogen compound D) were used as examples of the organic nitrogen compounds in accordance with the present teachings.

The effect of combining theanine with organic nitrogen compounds A to D was investigated by the following method.

### (1) Preparation of Theanine Solutions Containing Organic Nitrogen Compounds

Each of the organic nitrogen compounds A to D was respectively added to a theanine solution (manufactured by Taiyo Kagaku Co., Ltd.: Suntheanine® U001) in the amounts of 0 (control), 1, 5, 10, 50, and 100 ppm.

More specifically, 1996 mL of deionized water was added to 4 g of theanine, and this solution was used as the theanine control solution (2 mg/mL theanine solution) in the following tests. The taste threshold for theanine was confirmed to be 1.5 mg/mL, so the concentration of theanine was set above 1.5 mg/mL in each of the sample solutions.

0.1 mg of each organic nitrogen compound was weighed out and 500 mL of theanine control solution was added to each to prepare a set of 100 ppm solutions for each organic nitrogen compound (meaning a ratio of 0.2 ug/mL of organic nitrogen compound to 2 mg/mL of theanine).

150 mL of theanine control solution was added to 150 mL of the 100 ppm solution of each organic nitrogen compound solution to prepare a set of 50 ppm solutions.

180 mL of theanine control solution was added to 20 mL of the 100 ppm solution of each organic nitrogen compound solution to prepare a set of 10 ppm solutions.

180 mL of theanine control solution was added to 20 mL of the 50 ppm solution of each organic nitrogen compound solution to prepare a set of 5 ppm solutions.

180 mL of theanine control solution was added to 20 mL of the 10 ppm solution of each organic nitrogen compound solution to prepare a set of 1 ppm solutions.

### (2) Sensory Evaluation Test

A sensory (taste) evaluation was conducted by six panelists. The three evaluation categories were: sweetness, bitterness, and umami flavor. The evaluation method involved determining whether a difference in the above three tastes could be discerned compared to a control (i.e. the above-described theanine control solution) that does not contain any of the organic nitrogen compounds that were tested. The criteria for judgment were set at the following five levels (−2: weaker than the control, −1: slightly weaker than the control, 0: no difference, +1: slightly stronger than the control, +2: stronger than the control).

In the statistical analysis, a significance level of 10% (p<0.10) was used as the threshold for statistical significance.

### (3) Test Results

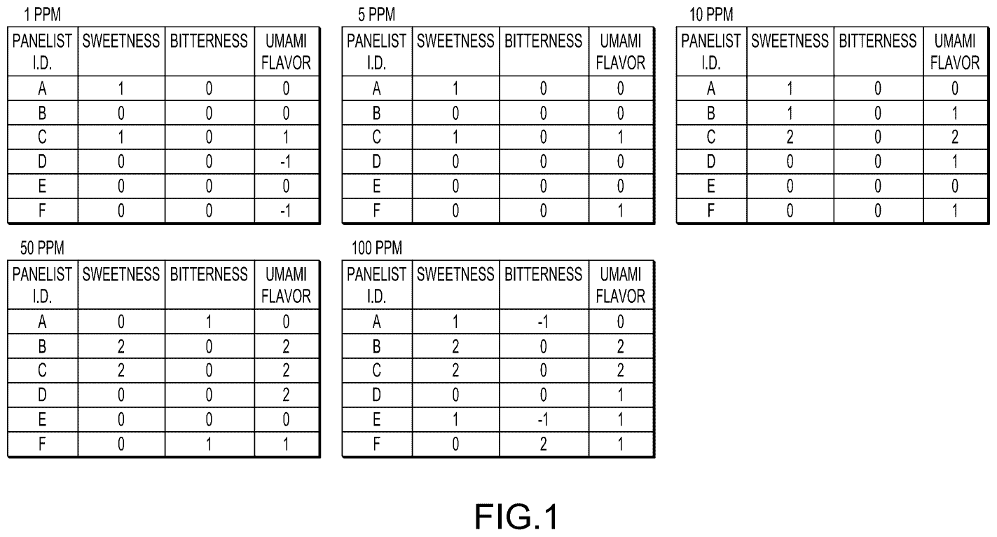

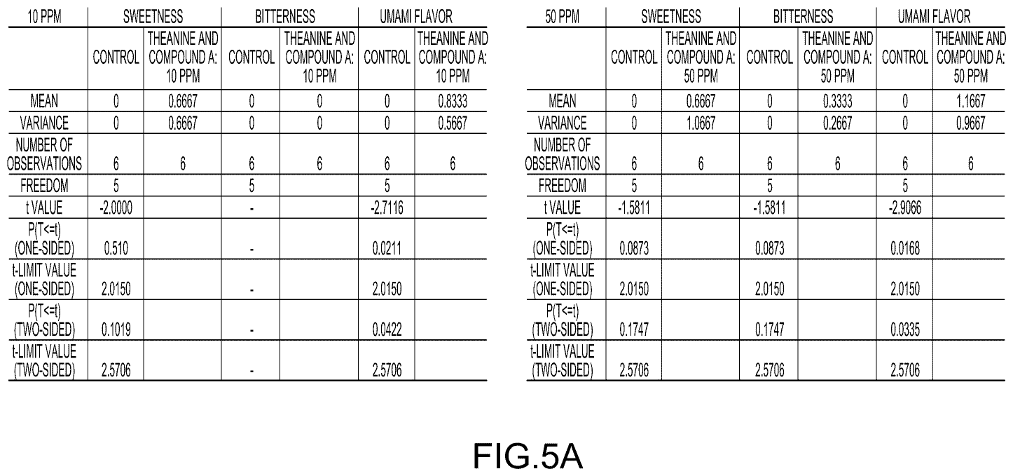

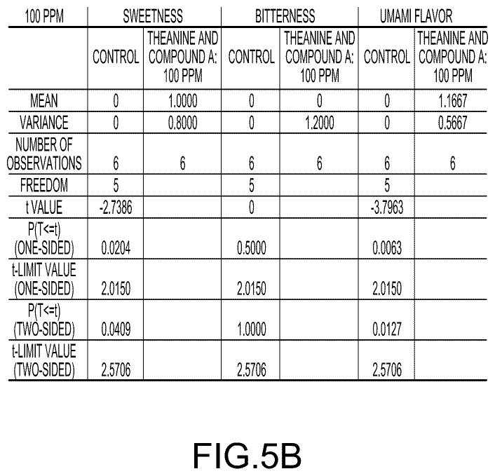

The results (raw data) of the tests of the various solutions containing the organic nitrogen compounds A to D are shown in FIGS. 1 to 4, respectively. The results of a Student's paired t-test for the organic nitrogen compounds A to C, respectively, based on the raw data shown in FIGS. 1-3, in which a significant difference in at least one evaluation category was discerned, are shown in FIGS. 5A to 7B.

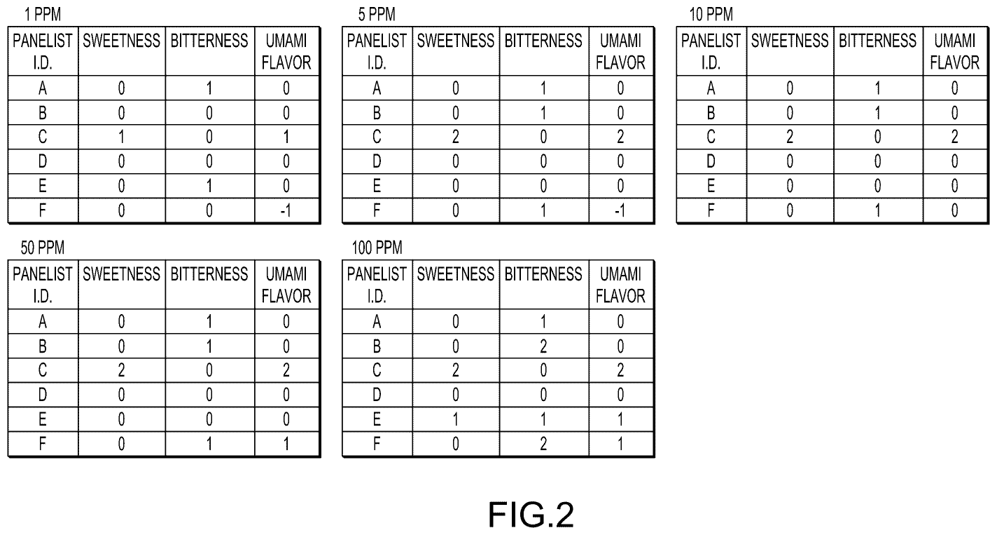

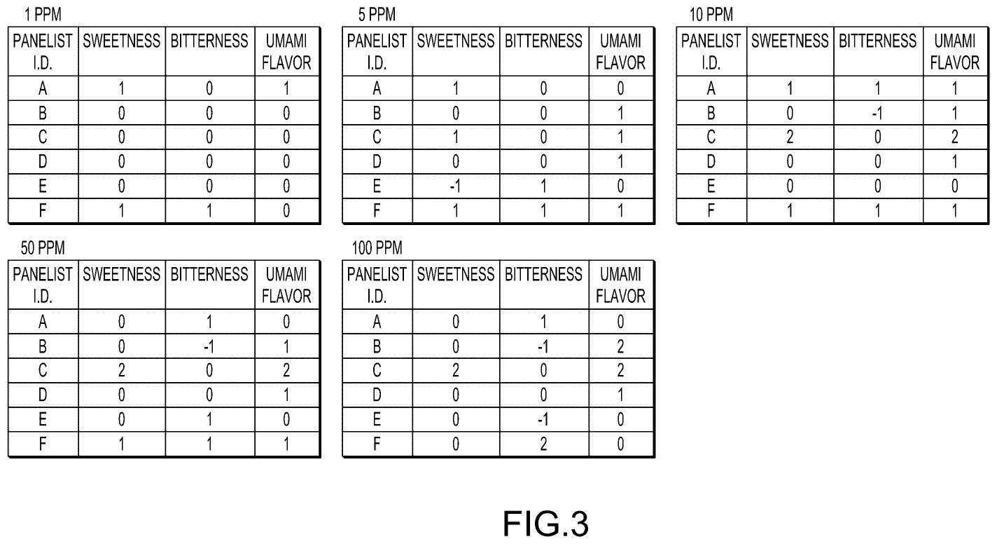

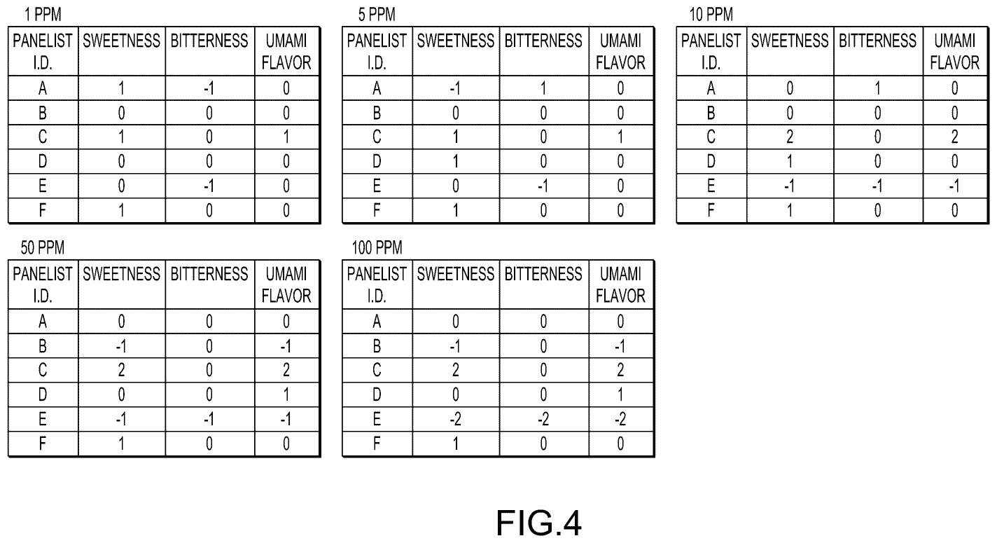

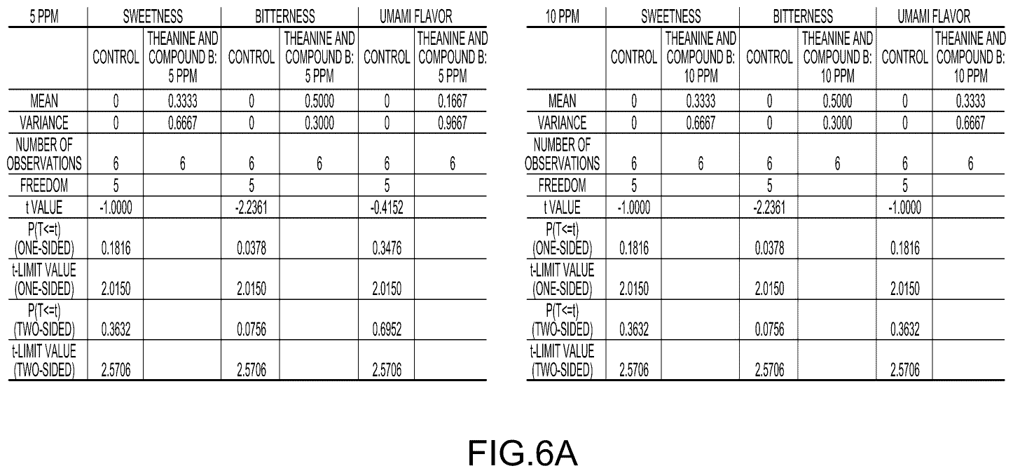

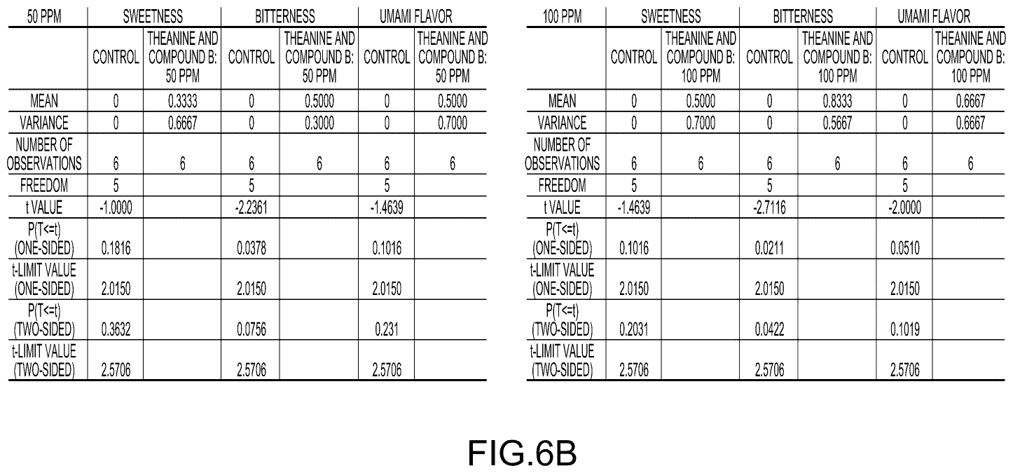

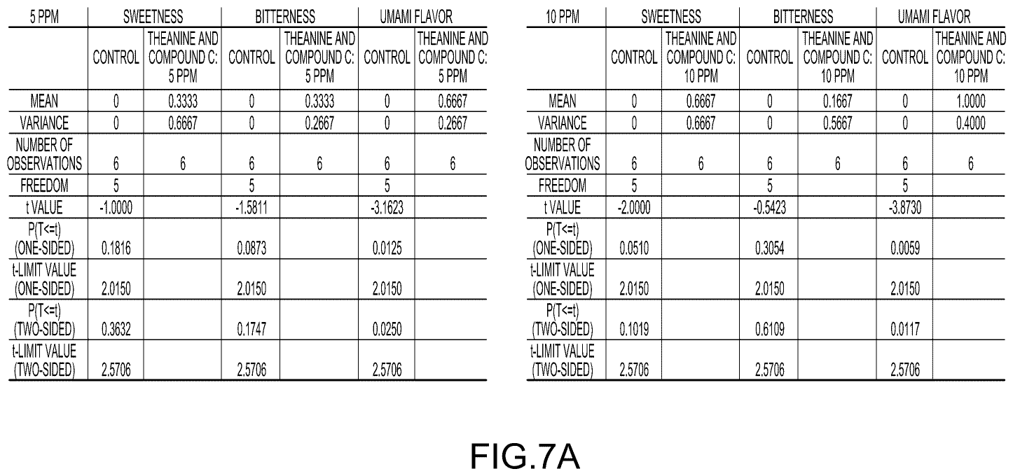

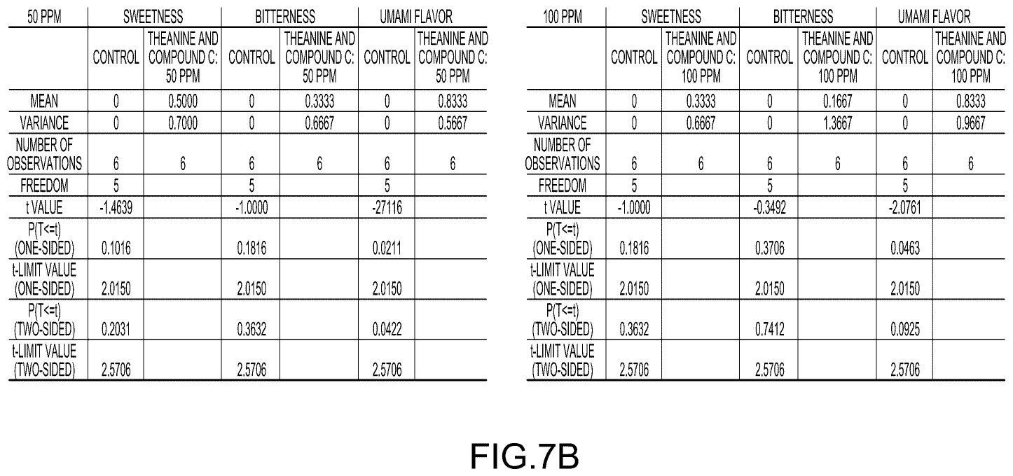

As was noted above, FIG. 1 shows the results of the sensory evaluation of solutions containing theanine and organic nitrogen compound A (ethylacetamide), FIG. 2 shows the results of the sensory evaluation of solutions containing theanine and organic nitrogen compound B (ethylformamide), FIG. 3 shows the results of the sensory evaluation of solutions containing theanine and organic nitrogen compound C (acetamide), FIG. 4 shows the results of the sensory evaluation of solutions containing theanine and organic nitrogen compound D (ethylpropionamide), FIGS. 5A and 5B show the analytical results of the sensory evaluation raw data shown in FIG. 1 for the solutions containing theanine and organic nitrogen compound A (ethylacetamide), FIGS. 6A and 6B show the analytical results of the sensory evaluation raw data shown in FIG. 1 for the solutions containing theanine and organic nitrogen compound B (ethylformamide), and FIGS. 7A and 7B show the analytical results of the sensory evaluation raw data shown in FIG. 1 for the solutions containing theanine and organic nitrogen compound C (acetamide).

The results shown in FIGS. 1-7B demonstrated that organic nitrogen compound A, ethylacetamide, had a significant effect (p<0.10) on umami flavor at concentrations of 10 ppm or more, and on sweetness at concentrations of 100 ppm or more. Organic nitrogen compound B, ethylformamide, had a significant effect (p<0.10) on bitterness at concentrations of 5 ppm or more, and organic nitrogen compound C, acetamide, had a significant effect (p<0.10) on umami flavor at concentrations of 5 ppm or more.

### Test Example 2: Evaluation of the Synergistic Effects of Ethylacetamide and Acetamide

Ethylacetamide (organic nitrogen compound A) and acetamide (organic nitrogen compound C) were used as the organic nitrogen compounds in this test. The synergistic effect on taste by adding both organic nitrogen compounds A and C to theanine solutions was investigated by the following method.

### (1) Preparation of Theanine Solutions Containing Organic Nitrogen Compounds

Solutions were prepared by adding equal amounts of organic nitrogen compounds A and C to a solution containing theanine (Taiyo Kagaku Co., Ltd.: Suntheanine® U001), to provide 0 (control), 1, 5, 10, 50, and 100 ppm solutions.

More specifically, 1996 mL of deionized water was added to 4 g of theanine, and this solution was used as the theanine control solution (2 mg/mL theanine solution).

Then, 0.1 mg of both of organic nitrogen compound A and C was added to 500 mL of the theanine control solution, to prepare a 100 ppm solution. The preparation of 50, 10, 5, and 1 ppm solutions was carried out in the same manner as in Test Example 1 above.

### (2) Sensory Evaluation Test

A sensory (taste) evaluation was conducted by six panelists. The three evaluation categories were again: sweetness, bitterness, and umami flavor. The evaluation method involved determining whether differences in the above three taste categories could be discerned compared to a control (i.e. the theanine control solution) that does not contain any organic nitrogen compounds. The criteria for judgment were again set at the following five levels (−2: weaker than the control, −1: slightly weaker than the control, 0: no difference, +1: slightly stronger than the control, +2: stronger than the control).

In the statistical analysis, a significance level of 10% (p<0.10) was used as the threshold for statistical significance.

### (3) Test Results

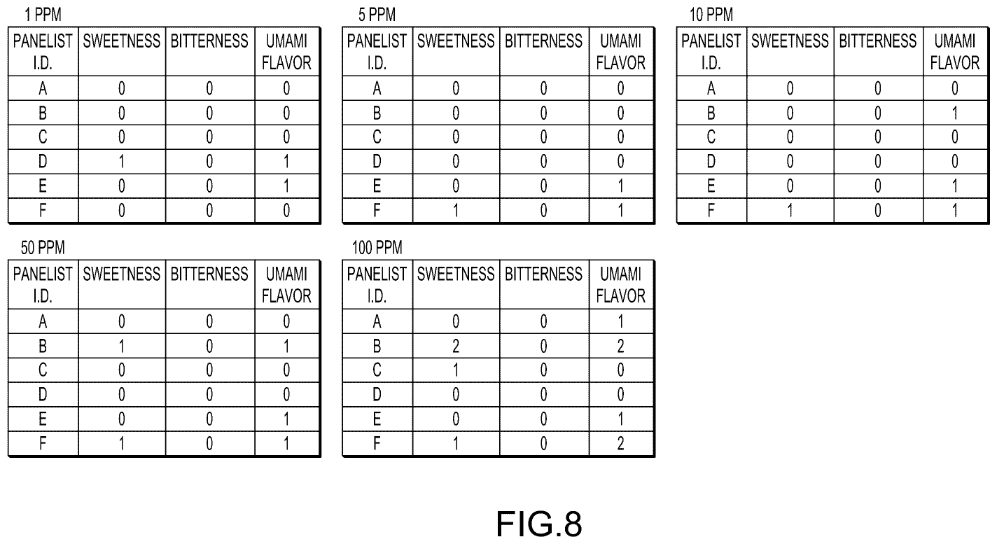

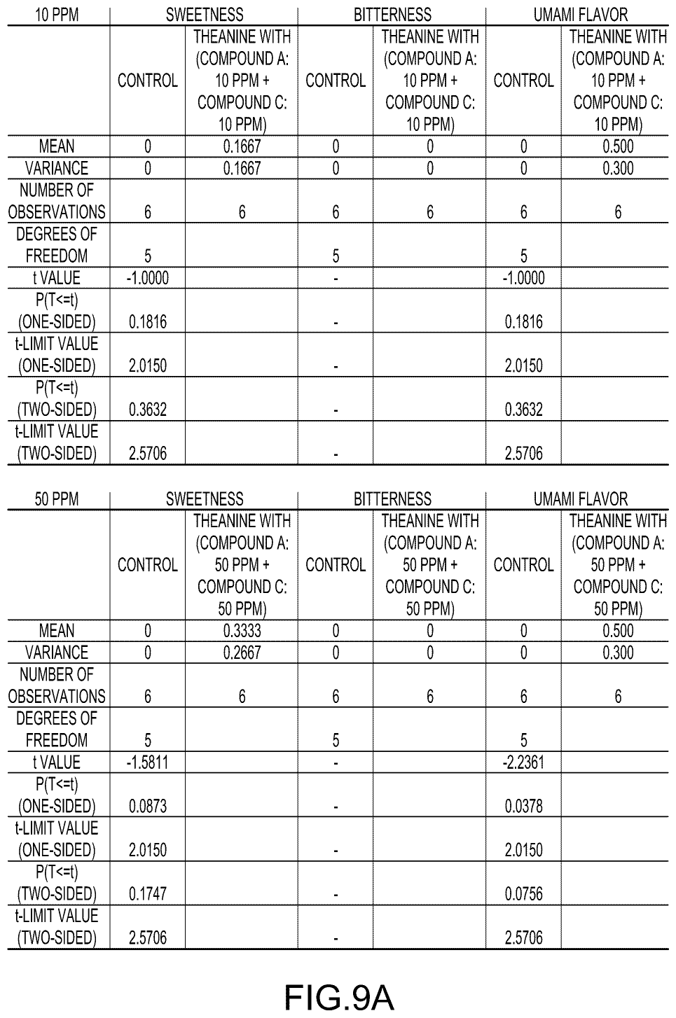

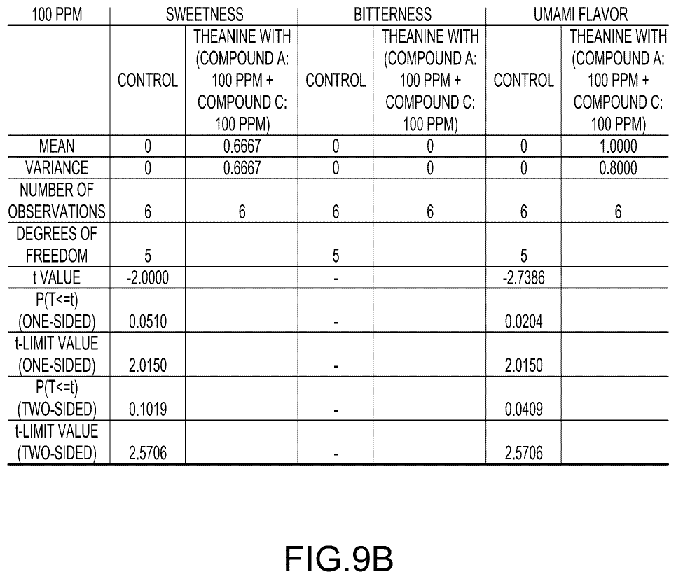

FIG. 8 shows the results (raw data) of the sensory evaluation of organic nitrogen compounds A and C in combination. FIGS. 9A and 9B show the results of a Student's paired t-test on the raw data shown in FIG. 8, in which a significant difference in at least one evaluation category was discerned.

These results demonstrated that the combination of ethylacetamide and acetamide with theanine had a significant effect on umami flavor (p<0.10) at concentrations of 10 ppm or more.

### Test Example 3: Evaluation of the Effects of Ethylacetamide and Acetamide (Confirmation of Upper Limit Value)

Ethylacetamide (organic nitrogen compound A) or acetamide (organic nitrogen compound C) was used as the organic nitrogen compound. The effect of combining theanine with organic nitrogen compounds A or C at high concentrations on a physiologically unacceptable unpleasant taste was investigated using the following method.

### (1) Preparation of Theanine Solutions Containing Organic Nitrogen Compounds

Solutions containing theanine (Taiyo Kagaku Co., Ltd.: Suntheanine (registered trademark) U001) were prepared to contain 0 (control), 100, 500, 1000, 5000, and 10000 ppm of organic nitrogen compound A or C.

More specifically, 1996 mL of deionized water was added to 4 g of theanine, to prepare the theanine control solution (2 mg/mL theanine solution). 10000 ppm organic nitrogen compound solutions were prepared by adding 500 mL of theanine control solution to 10 g of each organic nitrogen compound A and C.

150 mL of theanine control solution was added to 150 mL of each of the 10000 ppm organic nitrogen compound solutions to prepare 5000 ppm organic nitrogen compound solutions.

180 mL of theanine control solution was added to 20 mL of each of 10000 ppm organic nitrogen compound solutions to prepare 1000 ppm organic nitrogen compound solutions.

180 mL of theanine control solution was added to 20 mL of each of the 5000 ppm organic nitrogen compound solutions to prepare 500 ppm organic nitrogen compound solutions.

180 mL of theanine control solution was added to 20 mL of each of the 1000 ppm organic nitrogen compound solutions to prepare 100 ppm organic nitrogen compound solutions.

Similar solutions were also prepared for organic nitrogen compound C.

### (2) Sensory Evaluation Test

A sensory (taste) evaluation was conducted by six panelists. The evaluation category was unpleasant taste, which was considered to be an off-flavor. Each panelist evaluated whether there was a difference in off-flavor at each concentration compared to the control solution that did not contain any organic nitrogen compounds that were tested, and marked the concentration at which an unpleasant taste was observed with a “◯”.

### (3) Test Results

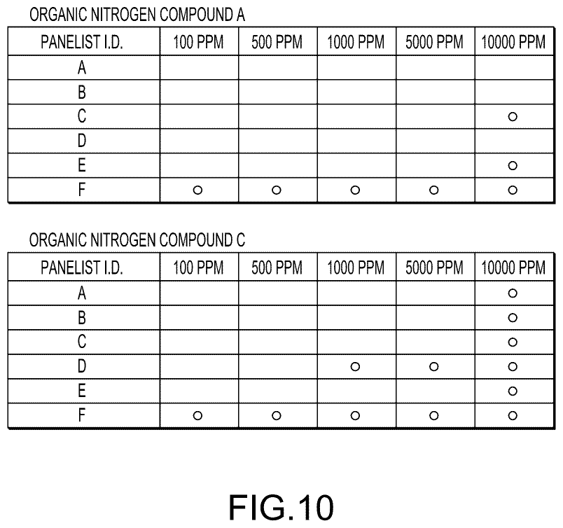

FIG. 10 shows the evaluation results for organic nitrogen compound A and C, respectively. For organic nitrogen compound A, one of six panelists noticed an unpleasant taste at concentrations of 100-5000 ppm, but at a concentration of 10000 ppm, three of six panelists noticed an unpleasant taste. For organic nitrogen compound C, one of six panelists noticed an unpleasant taste at concentrations of 100-500 ppm, two of six panelists noticed an unpleasant taste at concentrations of 1000-5000 ppm, but all panelists noticed an unpleasant taste at a concentration of 10000 ppm. The effect of organic nitrogen compound A and C on the unpleasant taste was acceptable (tolerated) up to a concentration of 5000 ppm.

### Test Example 4: Evaluation of the Effect of Ethylformamide Using a Three-Point Discrimination Method

The effect of using only ethylformamide (organic nitrogen compound B) without theanine as an organic nitrogen compound was examined using the following method to verify whether the significant difference in bitterness observed with the combination of theanine and ethylformamide was due to the combination of theanine and ethylformamide or due to ethylformamide alone.

### (1) Preparation of Test Solutions

Organic nitrogen compound B was added to deionized water (i.e. not containing theanine) to prepare solutions containing 0 (control), 5, 50, and 100 ppm of organic nitrogen compound B.

In other words, 0.1 mg of organic nitrogen compound B was dissolved in 1000 mL of deionized water (control) to make a 100 ppm solution. 150 mL of deionized water was added to 150 mL of the 100 ppm organic nitrogen compound B solution to make a 50 ppm organic nitrogen compound B solution.

20 mL of the 50 ppm organic nitrogen compound B solution was mixed with 180 mL of deionized water to make a 5 ppm organic nitrogen compound B solution.

### (2) Sensory Evaluation Test

A three-point discrimination sensory evaluation was conducted by eight panelists. Bitterness was used as the evaluation category. Two controls (deionized water) that did not contain organic nitrogen compound B were combined with a 5 ppm test solution. The panelists were asked to select solution(s) that they felt was (were) bitter. Analysis was performed using a binomial test. In the statistical analysis, a significance level of 10% (p<0.10) was used as the threshold for statistical significance.

### (3) Test Results

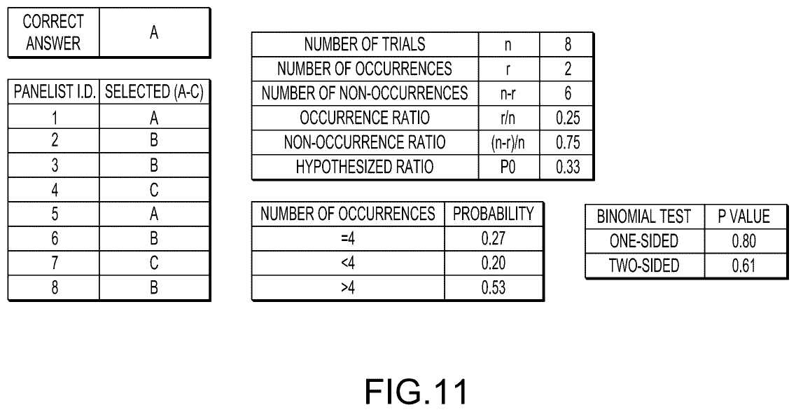

FIG. 11 shows the results of the three-point discrimination sensory evaluation. The eight panelists verified the results. The p-value was greater than the significance level, resulting in “no significant difference”. Thus, the addition of ethylformamide alone did not result in the presence or absence of bitterness, and it was therefore concluded that the effect of ethylformamide on the flavor was caused by its combination with theanine.

In addition, a sensory evaluation of the umami flavor and sweetness of ethylacetamide (organic nitrogen compound A) and of the umami flavor of acetamide (organic nitrogen compound C) had similar results to those of ethylformamide (organic nitrogen compound B).

As described above, these embodiments provide a composition that enhances the taste of theanine. Compositions according to the present teachings can be added to various foods and beverages to improve the taste thereof.

1. A theanine-containing composition containing (A) theanine and (B) an organic nitrogen compound.

2. The theanine-containing composition according to claim 1, in which (B) the organic nitrogen compound is at least one compound selected from the group consisting of amide compounds, imide compounds, and primary amines.

3. The theanine-containing composition according to claim 1, in which (B) the organic nitrogen compound is at least one compound selected from the group consisting of ethylformamide, ethylacetamide, and acetamide.

4. The theanine-containing composition according to claim 3, wherein:
(B) the organic nitrogen compound is ethylformamide, and
the ratio of ethylformamide to theanine is 5 ppm to 5000 ppm.

5. The theanine-containing composition according to claim 3, wherein:
(B) the organic nitrogen compound is ethylacetamide, and
the ratio of ethylacetamide to theanine is 5 ppm to 5000 ppm.

6. The theanine-containing composition according to claim 3, wherein:
(B) the organic nitrogen compound is acetamide, and
the ratio of acetamide to theanine is 5 ppm to 5000 ppm.

7. A food or beverage containing the theanine-containing composition according to claim 1.

8. A method for adding a new taste to a theanine-containing food, comprising:
adding an organic nitrogen compound to the theanine-containing food.

9. The method according to claim 8, wherein the organic nitrogen compound is at least one compound selected from the group consisting of amide compounds, imide compounds, and primary amines.

10. The method according to claim 8, in which the organic nitrogen compound is at least one compound selected from the group consisting of ethylformamide, ethylacetamide, and acetamide.

11. The method according to claim 10, wherein:
the organic nitrogen compound is ethylformamide, and
the ratio of ethylformamide to theanine is 5 ppm to 5000 ppm.

12. The method according to claim 10, wherein:
the organic nitrogen compound is ethylacetamide, and
the ratio of ethylacetamide to theanine is 5 ppm to 5000 ppm.

13. The method according to claim 10, wherein:
(B) the organic nitrogen compound is ethylacetamide, and
the ratio of ethylacetamide to theanine is 5 ppm to 5000 ppm.
